(19) United States
(12) Patent Application Publication
(10) Pub. No.: US 2024/0016629 A1
(43) Pub. Date: Jan. 18, 2024

(54) Powered Knee and Ankle Joint System with Adaptive Control

(71) Applicant: UNIVERSITY OF UTAH RESEARCH FOUNDATION, Salt Lake City, UT (US)

(72) Inventors: Tommaso LENZI, Salt Lake City, UT (US); Sarah HOOD, Salt Lake City, UT (US); Lukas GABERT, Salt Lake City, UT (US)

(21) Appl. No.: 18/032,924

(22) PCT Filed: Oct. 20, 2021
(86) PCT No.: PCT/US2021/055894

§ 371 Date: Apr. 20, 2023

Related U.S. Application Data
(60) Provisional application No. 63/094,220, filed on Oct. 20, 2020.

(51) Int. Cl. A61F 2/64; A61F 2/66; G16H 40/63

(52) U.S. Cl. CPC A61F 2/64; A61F 2/6607; G16H 40/63; A61F 2002/704

(57) ABSTRACT

A powered joint system that is configured to adaptively control powered joint movement during movement tasks includes a knee joint, one or more sensors, and a controller. The one or more sensors are configured to capture sensor data associated with a residual limb of a user. The controller comprises one or more processors and one or more hardware storage devices storing instructions that are executable by the one or more processors to configure the controller to perform various acts, including to: obtain a thigh orientation term, a thigh angular velocity term, and a thigh vertical acceleration term based on the sensor data; determine a target knee angle based on the thigh orientation term, the thigh angular velocity term, and the thigh vertical acceleration term; and output a signal configured to cause the knee joint to move toward the respective target joint angles.

### CROSS-REFERENCE TO RELATED APPLICATIONS

This application claims priority to U.S. Provisional Patent Application Ser. No. 63/094,220, filed Oct. 20, 2020 and titled “Powered Knee and Ankle Prosthesis With Adaptive Control”, the entirety of which is incorporated herein by this reference.

### STATEMENT REGARDING FEDERALLY SPONSORED RESEARCH

This invention was made with government support under grant no. R01HD098154 awarded by the National Institutes of Health. The government has certain rights in this invention.

### BACKGROUND

Conventional knee and ankle prostheses cannot provide net-positive energy, which is necessary to propel the body forward and upward during ambulation. Additionally, they cannot actively control the joint movements, which can be critical, for example, to achieve toe clearance in swing. While walking, individuals with an above-knee amputation make up for the deficiencies in their passive prostheses by performing undesirable compensatory movements with their residual limb, intact leg, and upper body. Unfortunately, these compensatory movements are insufficient for most users to ascend stairs in a step-over-step manner. As a result, individuals with a conventional passive prosthesis commonly ascend stairs using a slower and less efficient step-by-step gait pattern, leading each step with their intact leg. With this step-by step pattern, the intact leg and upper body performs all the effort required to climb the step, which requires significant strength and endurance. Moreover, the residual limb hip joint needs to extend and circumduct unnaturally for the passive prothesis to clear the step during swing as the prosthetic knee joint cannot flex as the biological leg would. This residual limb extension is often difficult due to muscle contractures, further challenging the user's balance.

Accordingly, there is an ongoing need for improved prosthesis systems to enable individuals with above-knee amputations to ascend stairs more naturally.

The subject matter claimed herein is not limited to embodiments that solve any disadvantages or that operate only in environments such as those described above. Rather, this background is only provided to illustrate one exemplary technology area where some embodiments described herein may be practiced.

### SUMMARY

Disclosed embodiments include a powered prosthesis that is configured to adaptively control powered knee and ankle joint movements during climbing tasks. The powered prosthesis includes a knee joint and an ankle joint, one or more sensors, and a controller. The one or more sensors are configured to capture sensor data associated with a residual limb of a user. The controller comprises one or more processors and one or more hardware storage devices storing instructions that are executable by the one or more processors to configure the controller to perform various acts, including to: obtain a thigh orientation term, a thigh angular velocity term, and a thigh vertical acceleration term based on the sensor data; determine target knee and ankle angles based on the thigh orientation term, the thigh angular velocity term, and the thigh vertical acceleration term; and output a signal configured to cause the knee and ankle joints to move toward the target knee and ankle joint angles.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered limiting in scope, embodiments will be described and explained with additional specificity and detail through the use of the accompanying drawings.

FIG. 1 illustrates a conceptual representation of operation of an adaptive stair controller, in accordance with the present disclosure;

FIG. 2 illustrates a perspective view of example components of an example powered knee and ankle prosthesis;

FIG. 3 illustrates an example graph depicting a relationship between thigh position and powered prosthesis knee joint desired position;

FIG. 4 illustrates an example graph depicting a relationship between thigh velocity and powered prosthesis knee joint desired position;

FIGS. 5 and 6 illustrate example graphs depicting relationships between thigh vertical acceleration and powered prosthesis knee joint desired position;

FIG. 7 illustrates an example graph depicting a relationship between thigh position and powered prosthesis ankle joint desired position;

FIGS. 8 and 9 illustrate example graphs depicting relationships between thigh vertical acceleration and powered prosthesis ankle joint desired position;

FIG. 10 illustrates an example graph depicting a relationship between knee position at the start of a Standing state with respect to peak knee torque;

FIG. 11 illustrates an example graph depicting a relationship between knee position at the start of a Standing state with respect to knee position at peak torque;

FIG. 12 illustrates an example graph depicting desired torque as a function of (i) the measured knee angle at the transition between a Lifting state and a Standing state and (ii) a currently measured knee angle;

FIG. 13 illustrates an example block diagram depicting closed-loop position controllers usable during a Lifting state;

FIG. 14 illustrates an example block diagram depicting a virtual impedance controller for defining a desired torque command during a Standing state;

FIG. 15 illustrates an example block diagram depicting a low-level torque controller for implementing a desired torque command;

FIG. 16 illustrates an example flow diagram depicting acts associated with adaptively controlling powered joint movement during climbing tasks, in accordance with the present disclosure;

FIG. 17 illustrates an example graph depicting the swing trajectory of a powered prosthesis from cartesian space for different gait patterns and stair heights;

FIG. 18 illustrates an example graph depicting the duration of swing for different gait patterns and stair heights;

FIG. 19 illustrates an example graph depicting kinematic analysis of the thigh segment, knee joint, and ankle joint for different gait patterns and stair heights;

FIGS. 20 and 21 illustrate example graphs depicting kinematic analysis of a standing phase for different gait patterns and stair heights; and

FIG. 22 illustrates an example graph depicting kinematic analysis of a thigh segment, knee joint, and ankle joint for a powered prosthesis and for a sound human limb.

### DETAILED DESCRIPTION

### Overview

Many challenges exist for individuals with above-knee amputations, including the ascension of stairs. Because passive prostheses cannot actively control the joint movements, individuals with above-knee amputations who rely on a passive prosthesis typically perform compensatory movements with their residual limb and upper body to ascend stairs in a step-by-step pattern (rather than a step-over-step pattern).

Powered prostheses have the potential to imitate the biological leg biomechanics during stair ascent. A powered prosthesis can propel the body upward by injecting positive energy when the prosthetic foot is in contact with the step (i.e., during the stance phase, also referred to herein as the standing phase). Also, a powered prosthesis can ensure adequate clearance with the step and correctly place the prosthetic foot in preparation for the next step to be climbed by actively controlling the joint movements when the prosthetic foot is off the ground (i.e., during the swing phase, also referred to herein as the lifting phase). A powered prosthesis may thus improve stairs ambulation speed and/or reduced metabolic effort compared to conventional passive prostheses.

There are many challenges associated with implementing powered prostheses for stair ascension in real-world scenarios. For example, because climbing taller steps requires larger net-positive energy and higher joint torque than climbing smaller steps, the torque generated by the prosthesis in stance phase should be adapted to the step height in order to accommodate different step heights that users may encounter. In addition, different step heights or variations in gait patterns may require the prosthesis to change the swing movement trajectory so that proper clearance and foot placement on the step can be achieved. Thus, to be practical for real-world implementation, powered prosthesis controllers must be robust to variability in stair geometry, gait pattern, and gait cadence to enable stair ascension.

Although powered prostheses show promise for enabling above-knee amputee subjects to ascend stairs step-over-step, available stair ascent controllers are designed to produce a predefined, fixed action of the powered prothesis, which must be manually tuned for each subject and staircase. Thus, if a user attempts to climb a taller step than the one the stair controller was tuned for, the prosthesis may not provide enough clearance, which may cause the prosthetic foot to hit the step riser and may result in user injury. Conversely, if the user attempts to climb a shorter step than the one the stair controller was tuned for, the prosthesis may provide too much clearance, which may cause user imbalance upon landing on the step. Furthermore, even if the step is cleared by the user through hip circumduction and sound-side vaulting, the prosthetic foot may fail to lay flat on the step. Subsequently, the prosthetic knee may begin to generate torque (to climb a subsequent step) while the prosthetic foot fails to lay flat on the step, which may result in the subject being pushed backward rather than upward and may potentially cause the user to fall.

Thus, at least one aspect of the present disclosure is to provide powered prosthesis controllers that automatically adapt to the variability of different stair heights. Such controllers may be implemented in real-world environments, where users may encounter steps of different heights.

In contrast with existing approaches, the present disclosure provides an alternative control strategy for a powered knee and ankle prosthesis to ascend stairs in a manner that accounts for varying step heights, cadences, and/or gait patterns. For example, embodiments of the present disclosure modulate the prosthesis knee and ankle position in swing as a continuous function of the user's thigh position, thigh velocity, and/or thigh vertical acceleration. In some instances, disclosed embodiments modulate energy injection in stance using a continuous adaption of knee joint torque-angle relationship as a function of the prothesis knee position when the prosthetic foot contacts the step.

By implementing the disclosed principles, stance energy and/or swing trajectory may be continuously changed or modulated during stair ambulation (in contrast with existing approaches, which have relied on the residual limb orientation as a proxy for gait phase to produce a fixed prosthesis trajectory in level-ground walking). Disclosed embodiments may enable individuals with above-knee amputations to climb stairs of different heights at different cadences and to seamlessly transition between different stair climbing strategies (e.g., step-by-step, step-over-step, two-step, etc.). Disclosed embodiments may thus facilitate the implementation of powered prostheses for stair ascension in real-world environments.

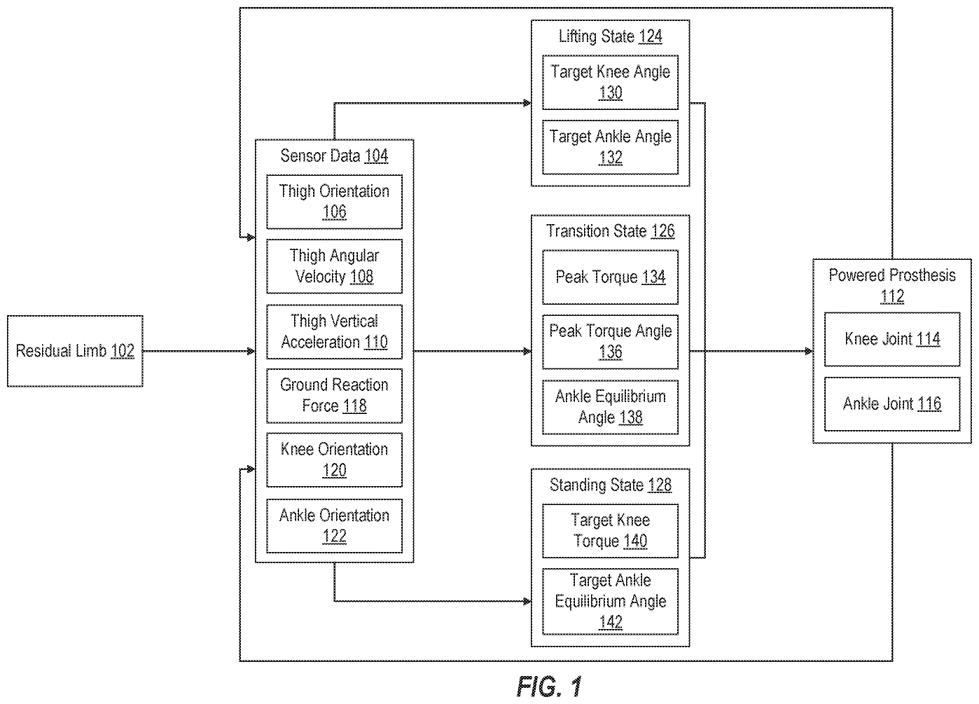

FIG. 1 illustrates a conceptual representation of operation of an adaptive stair controller, in accordance with the present disclosure. FIG. 1 depicts a residual limb 102, which may comprise a residual limb of an above-the-knee amputee. Various sensors may be deployed relative to the residual limb to obtain sensor data associated with the residual limb. Accordingly, FIG. 1 depicts sensor data 104, which includes various measurable values related to the residual limb 102. In particular, FIG. 1 illustrates the sensor data 104 as indicating a thigh orientation 106, a thigh angular velocity 108, and a thigh vertical acceleration 110. As will be described in more detail hereinafter, these measured values related to the residual limb 102 may be used to facilitate adaptive control of a powered prosthesis 112 for stair ascent, which may include a knee joint 114 and an ankle joint 116.

FIG. 1 also illustrates that the sensor data 104 may indicate additional measurements related to one or more components of a powered prosthesis 112. For example, the sensor data 104 may comprise or indicate a ground reaction force 118, a knee orientation 120 (e.g., of the knee joint 114 of the powered prosthesis 112), and/or an ankle orientation 122 (e.g., of the ankle joint 116 of the powered prosthesis 112). As will be described in more detail hereinafter, such measurements may be used to facilitate adaptive control of the powered prosthesis 112 for stair ascent.

By way of overview, the sensor data 104 may be utilized to dynamically determine a state within which to operate/actuate the powered prosthesis. For example, based on the ground reaction force 118, a lifting state 124, a standing state 128, or a transition state 126 (e.g., a transition from the lifting state to the standing state 128) may be detected or selected. When in the lifting state 124, various sensor data 104 (e.g., the thigh orientation 106, the thigh angular velocity 108, and/or the thigh vertical acceleration 110) may be used to determine a target knee angle 130 and/or a target ankle angle 132. The target knee angle 130 may be used to control actuation of the knee joint 114 of the powered prosthesis, and the target ankle angle 132 may be utilized to control actuation of the ankle joint 116 of the powered prosthesis.

When in the transition state 126, various sensor data 104 may be used to determine a peak torque 134, a peak torque angle 136, and/or an ankle equilibrium angle 138. The peak torque 134 and the peak torque angle 136 may be used to control actuation of the knee joint 114 during the standing state, and the ankle equilibrium angle 138 may be used to control actuation of the ankle joint 116 during the standing state.

When in the standing state 128, various sensor data 104 may be used to determine a target knee torque 140 and/or a target ankle equilibrium angle 142, which may be used to control actuation of the knee joint 114 and the ankle joint 116, respectively.

Updated sensor data may be continuously obtained to facilitate adaptive modification of the values generated/determined to control actuation of the knee joint 114 and the ankle joint 116 (e.g., the target knee angle 130, the target ankle angle 132, the peak torque 134, the peak torque angle 136, the ankle equilibrium angle 138, the target knee torque 140, the target ankle equilibrium angle 142, etc.). In this way, powered prosthesis controllers may adapt to variable stair height, user cadences, and/or user gait patterns.

Although the examples discussed in the present disclosure focus, in at least some respects, on adaptive control of a powered joint system implemented as a powered prosthesis (e.g., for above-knee amputees), the principles disclosed herein related to adaptive control may be applied to controllers of other types of powered joint systems, such as powered exoskeleton systems (e.g., powered knee and/or powered ankle exoskeletons that include knee and/or ankle joints). Furthermore, although examples discussed herein focus, in at least some respects, on stair climbing, one will appreciate, in view of the present disclosure, that the disclosed principles may be utilized for other movement tasks, such as squatting, lunging, sit-to-stand transferring, and/or others.

Having described some of the various high-level features and benefits of the disclosed embodiments, attention will now be directed to FIGS. 1 through 22. These Figures illustrate various supporting illustrations related to the disclosed embodiments.

### Example Powered Knee and Ankle Prosthesis

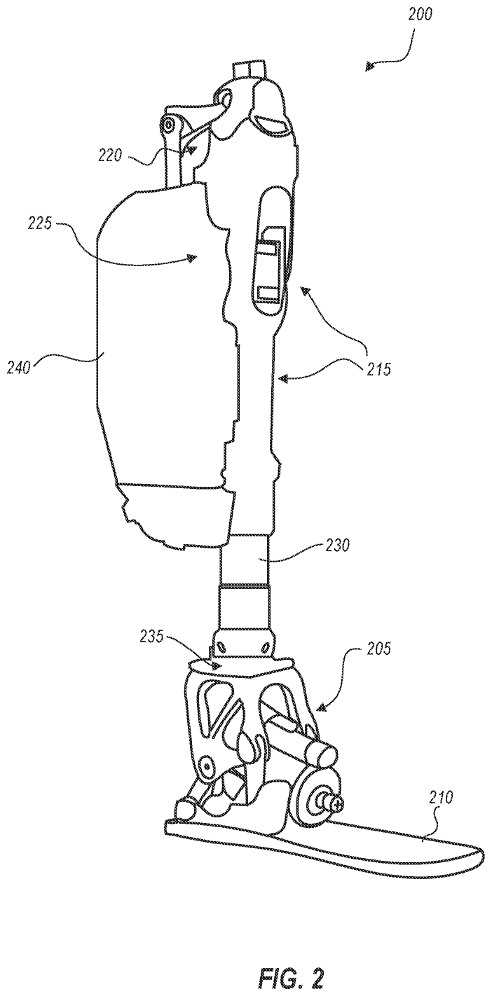

Systems, methods, and techniques related to adaptive stair controllers for knee and ankle prostheses, in accordance with the present disclosure, may be implemented utilizing various types of knee and ankle prostheses. FIG. 2 illustrates a perspective view of an example powered knee and ankle prosthesis 200 that may be implemented in conjunction with the principles disclosed herein related to shared neural controllers. One will appreciate, in view of the present disclosure, that the particular components and/or features of the powered knee and ankle prosthesis 200 of FIG. 2 do not limit the applicability of the disclosed principles related to shared neural controllers to other types of powered knee and ankle prostheses that include additional or alternative components.

The example powered knee and ankle prosthesis 200 of FIG. 2 comprises a self-contained, battery-operated, powered knee and ankle prosthesis that can generate biologically appropriate torque and power during ambulation. The powered knee and ankle prosthesis 200 of FIG. 2 may be configured and/or adjustable to fit users associated with various body sizes. For example, the powered knee and ankle prosthesis 200 may be sized to fit the 50th percentile female leg profile. The powered knee and ankle prosthesis 200 may comprise any suitable weight, such as within a range of about 1.5 kg to about 8 kg (e.g., about 2.5 kg with the battery and protective covers included).

The example powered knee and ankle prosthesis 200 of FIG. 2 comprises an ankle-foot module 205. The ankle-foot module 205 may utilize a compact, lightweight powered polycentric design, which may be contained within a commercially available foot shell. The powered polycentric mechanism of the ankle-foot module 205 may be connected/connectable to custom carbon-fiber feet 210 of different sizes to accommodate different subjects.

The example powered knee and ankle prosthesis 200 of FIG. 2 further comprises a knee module 215. The knee module 215 may utilize an active variable transmission 220 (AVT 220) to optimize the effective transmission ratio and leg dynamics for different locomotion tasks. In addition, the knee module 215 may contain/comprise a control unit and battery 225 and/or motor drivers for both the knee joint and the ankle joint. The knee module 215 and the ankle-foot module may connect with a pylon 230 (e.g., a standard 30-mm pylon), which may allow for height and intra-extra rotation adjustments. In some instances, a pyramid adapter 235 is implemented at the top of the ankle-foot module 205 to estimate the ground reaction force and torque.

The AVT 220 of the example powered knee and ankle prosthesis 200 of FIG. 2 utilizes a DC motor (e.g., a Maxon Motor EC 13, 18 V, 12 W) connected to a 4.1:1 planetary gear, which drives the nut on a bigger, non-backdrivable leadscrew (e.g., M4×1.25, single start) through a 1:1 spur gear transmission. In the example powered knee and ankle prosthesis 200 of FIG. 2, two thrust bearings and two ball bearings are used to support the leadscrew's axial and radial loads. In addition, the lead screw of the AVT 220 can be supported by two parallel guides realized by slotted cranks with dry bushings (e.g., IGUS® Iglidur® L280, static friction coefficient 0.23, dynamic friction coefficient 0.08-0.23). The slotted crank defines the range of motion of the AVT 220 (e.g., a range of motion within a range of about 20 mm to about 45 mm). The overall structural safety factor of the example powered knee and ankle prosthesis 200 represented in FIG. 2 is 2.5. An incremental encoder (e.g., RLS, RM08) is, in some instances, located on the spur gear to measure the position of the AVT 220. This sensor (i.e., the incremental encoder), together with a four-quadrant motor driver (e.g., Maxon Motor ES CON module 24/2) may enable feedback control of the position of the AVT 220 in both driving and braking operations. As noted above, other configurations are within the scope of the present disclosure.

The primary actuator of the example powered knee and ankle prosthesis 200 represented in FIG. 2 is a rotary-to-linear system comprising a brushless DC motor (e.g., Maxon Motor EC-4pole 22, 24 V, 120 W), a roller screw (e.g., Rollvis®, pitch diameter 4.5 mm, lead 2 mm, static-dynamic load ratings 7.2-7.8 kN, efficiency 90%), and a timing-belt transmission (e.g., 48:18 teeth ratio). The roller screw nut is supported by a linear guide (e.g., Helix Linear Technologies, HMR9ML, basic load/moment ratings 3880 N/12.4 Nm). The main motor can be located inside of an aluminium frame (e.g., 7075 T6-SN) which may also operate as a heatsink. A force-torque sensor is, in some instances, embedded in the pylon 230 to detect contact with the ground. Furthermore, in some implementations, one or more 9-DOF IMUs (MPU9250, Invsense) are included to sense the movements and the orientation of the leg in space. IMUs may be placed, for example, on the foot, shank, and thigh segments to track their movement in space.

Covers 240 (e.g., 3D printed covers) may be utilized to house the control unit and battery 225. The control unit and battery 225 may comprise a Li-Ion battery (e.g., 2500 mAh, 6S) and/or an onboard system-on-module (SOM) (e.g., myRIO 1900, National Instruments, 100 g without covers). The SOM can run all custom control algorithms in real time, interfacing with the sensors and servo drivers for the AVT 220 and the primary motor (e.g., Elmo, Gold Twitter G-TWI 30/60SE, 35 g). The SOM can be connected through wi-fi to a host computer, smartphone, and/or other device for data monitoring and/or controller tuning.

Experimental results (discussed in more detail hereinafter) were obtained by implementing an adaptive stair controller with a powered knee and ankle prosthesis 200 that includes the features/components discussed with reference to FIG. 2. However, as noted previously, the principles discussed herein related to adaptive stair controllers are not limited to the particular components/features of the powered knee and ankle prosthesis 200 discussed above with reference to FIG. 2.

### Example Adaptive Stair Controller Architecture

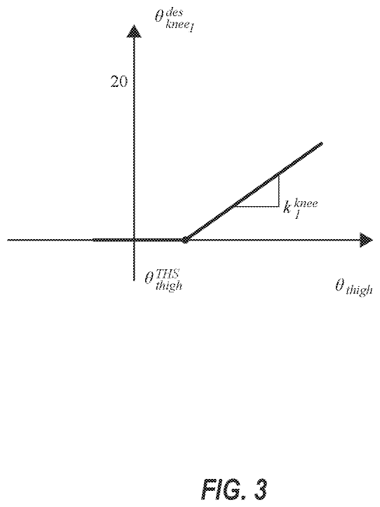

In accordance with the present disclosure, at a high-level, an adaptive stair controller utilizes a finite-state machine with two states: Standing and Lifting. The Lifting state may be configured to become active (or entered) in response to various triggering conditions. For example, in some instances, the adaptive stair controller may be in the Lifting state when the ground reaction force (GRF) is lower than a predefined threshold (GRF^{THS}) In Lifting, the desired angle of the knee joint (θₖₙₑₑ^{des}) (or target knee angle) and the desired angle of the ankle joint (θₐₙₖₗₑ^{des}) (or target ankle angle) can be continuously adapted based on the movements of the user's thigh (i.e., the thigh of the user's residual limb). To this end, in some embodiments, the target knee angle (θₖₙₑₑ^{des}) is defined as the sum of three terms: (θₖₙₑₑ_{₁}^{des}), (θₖₙₑₑ_{₂}^{des}), and (θₖₙₑₑ_{₃}^{des}), which may be determined utilizing Equations (1), (2), (3) and (4). The first term, (θₖₙₑₑ_{₁}^{des}) (also referred to herein as a thigh orientation term), is proportional to the orientation of the user's thigh with respect to gravity (θ_{thigh}) provided a predefined certain threshold (θ_{thigh}^{THS}) is exceeded, as defined in Equation (1) below and as illustrated in FIG. 3, which illustrates an example graph depicting a relationship between thigh position and powered prosthesis knee joint desired position.

$$\{ \begin{matrix}{θ}_{{knee}_{1}}^{des} = {k}_{1}^{knee} ( {θ}_{thigh} - {θ}_{thigh}^{THS} ) & ∀ {θ}_{thigh} \geq {θ}_{thigh}^{THS} \\ {θ}_{{knee}_{1}}^{des} = 0 & ∀ {θ}_{thigh} < {θ}_{thigh}^{THS}\end{matrix} \qquad ( 1 )$$

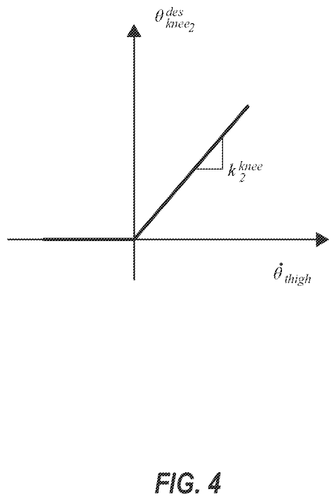

The second term (θₖₙₑₑ_{₂}^{des}) (also referred to herein as a thigh angular velocity term) is proportional to the positive angular velocity of the user's thigh (\dot{θ}_{thigh}) as defined in Equation (2) and shown FIG. 4, which illustrates an example graph depicting a relationship between thigh velocity and powered prosthesis knee joint desired position.

$$\{ \begin{matrix}{θ}_{{knee}_{2}}^{des} = {k}_{1}^{knee} {\dot{θ}}_{thigh} & ∀ {\dot{θ}}_{thigh} \geq θ \\ {θ}_{{knee}_{2}}^{des} = 0 & ∀ {\dot{θ}}_{thigh} < θ\end{matrix} \qquad ( 2 )$$

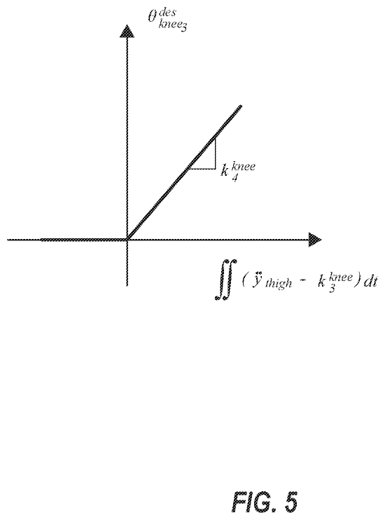

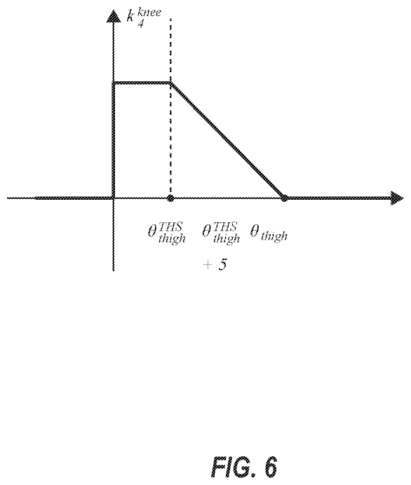

The third term (θₖₙₑₑ_{₃}^{des}) (also referred to herein as a thigh vertical acceleration term) depends on the vertical acceleration of the user's thigh with respect to gravity (ÿ_{thigh}). FIG. 5 illustrates an example graph depicting a relationship between thigh vertical acceleration and powered prosthesis knee joint desired position. As defined in Equation (3) and shown in FIG. 5, a first factor (k₃) is subtracted to the thigh acceleration (ÿ_{thigh}) before calculating the double integral. The result of the double integration is then multiplied by a second non-constant factor (k₄) as defined in Equation (4). This multiplication factor changes as a function of the thigh orientation (θ_{thigh}) as shown in FIG. 6, which illustrates another relationship between thigh vertical acceleration and powered prosthesis knee joint desired position. Specifically, the multiplication factor is kept constant (k₄^{0,knee}) until the thigh orientation (θ_{thigh}) exceeds a certain threshold (θ_{thigh}^{THS}). Above the threshold, the multiplication factor decreases linearly, reaching zero when the thigh orientation equals to the thigh threshold plus an offset (e.g., 5° in the example shown in FIG. 6: θ_{thigh}^{THS}+5°).

$${θ}_{{knee}_{3}}^{des} = {k}_{4}^{knee} \int \int ( {\ddot{y}}_{thigh} {k}_{3}^{knee} ) dt \qquad ( 3 )$$

$$\{ \begin{matrix}{k}_{4}^{knee} = {k}_{4}^{0 , knee} \\ ∀ ( {θ}_{thigh} \geq 0 \&\& {θ}_{thigh} < {θ}_{thigh}^{THS} \\ {k}_{4}^{knee} = {k}_{4}^{0 , knee} - \frac{{k}_{4}^{0 , knee} ( {θ}_{thigh} - {θ}_{thigh}^{THS} )}{5} \\ ∀ ( {θ}_{thigh} \geq {θ}_{thigh}^{THS} \&\& {θ}_{thigh} < {θ}_{thigh}^{THS} + 5 ) \\ {k}_{4}^{knee} = 0 \\ ∀ ( {θ}_{thigh} \geq {θ}_{thigh}^{THS} + 5 ‖ {θ}_{thigh} < 0 )\end{matrix} \qquad ( 4 )$$

In view of the foregoing, k₄ operates as a linear gain that decreases as the thigh orientation angle increases after a certain threshold has been achieved. With the disclosed methodology, the knee flexion position increases with the hip flexion angle, with faster hip flexion movement resulting in higher knee flexion angles. Moreover, in some instances, the prosthetic knee flexes whenever the foot is lifted from the floor even if the residual hip joint does not flex.

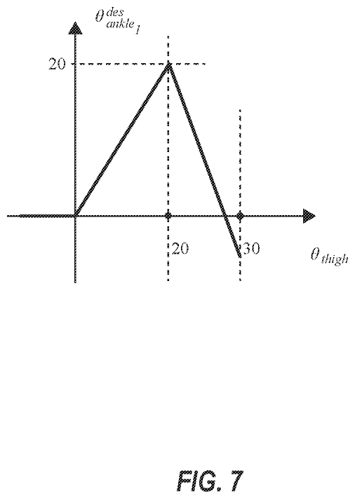

The desired angular position of the ankle joint (θₐₙₖₗₑ^{des}) (or target ankle angle) is the sum of two terms. The first term (θₐₙₖₗₑ_{₁}^{des}) (also referred to herein as a second thigh orientation term) depends on the thigh position as defined in Equation (5) below and shown in FIG. 7. In some implementations, this term (i.e., θₐₙₖₗₑ_{₁}^{des}) is zero for thigh angles lower than zero. When the thigh angle is between 0° and 20° (e.g., within a first range of thigh orientation angles, where between 0° and 20° is provided by way of example only), the desired ankle angle is proportional to the thigh orientation angle. For thigh angles between 20° and 30° (e.g., within a second range of thigh orientation angles, where between 20° and 30° is provided by way of example only), the desired ankle angle is linearly decreased to the ankle angle required to match the shank angle at 30°. For thigh angles greater than 30° (e.g., for thigh orientation angles above the second range of thigh orientation angles), the desired ankle angle is equal to the shank orientation angle, so that the prosthetic foot can remain perpendicular to gravity in order to stay parallel to the ground/step.

$$\{ \begin{matrix}{θ}_{{ankle}_{2}}^{des} = {k}_{4}^{ankle} ( {θ}_{thigh} - {θ}_{thigh}^{THS} ) \\ ∀ {θ}_{thigh} < 20 \\ {{θ}_{ankle}^{des}}_{1} = ( 20 + \frac{{θ}_{shank} - 20}{10} ) ( {θ}_{thigh} - 20 ) \\ ∀ ( {θ}_{thigh} > 20 \&\& {θ}_{thigh} < 30 ) \\ {{θ}_{ankle}^{des}}_{1} = {θ}_{shank} \\ ∀ {θ}_{thigh} > 30\end{matrix} > 30 \qquad ( 5 )$$

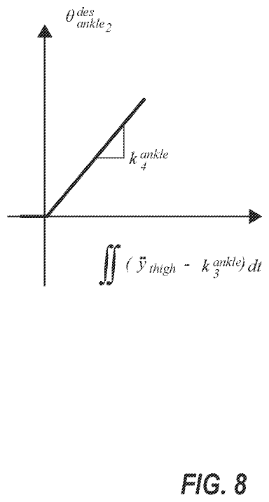

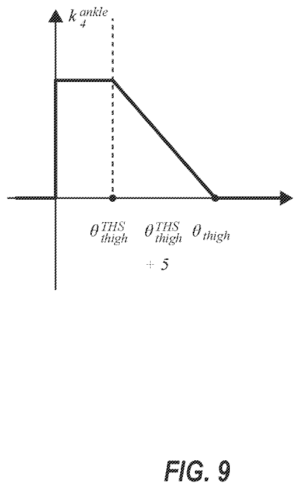

The second term of the desired ankle angle (θₐₙₖₗₑ_{₂}^{des}) (also referred to herein as a second thigh vertical acceleration term) depends on the vertical acceleration of the user's thigh with respect to gravity (ÿ_{thigh}) similarly to the knee joint (e.g., similar to the thigh vertical acceleration term associated with the target knee joint, described in Equation (3) and Equation (4)), although ankle-specific gains are used as described in Equation (6) and Equation (7) and shown in FIGS. 8 and 9.

$$\begin{matrix}{θ}_{{ankle}_{2}}^{des} = {k}_{4}^{ankle} \int \int ( {\ddot{y}}_{thigh} - {k}_{3}^{ankle} ) dt & ( 6 ) \\ \{ \begin{matrix}{k}_{4}^{ankle} = {k}_{4}^{0 , ankle} \\ ∀ ( {θ}_{thigh} \geq 0 \&\& {θ}_{thigh} < {θ}_{thigh}^{THS} \\ {k}_{4}^{ankle} = {k}_{4}^{0 , ankle} - \frac{{k}_{4}^{0 , ankle} ( {θ}_{thigh} - {θ}_{thigh}^{THS} )}{5} \\ ∀ ( {θ}_{thigh} \geq {θ}_{thigh}^{THS} \&\& {θ}_{thigh} < {θ}_{thigh}^{THS} + 5 ) \\ {k}_{4}^{ankle} = 0 \\ ∀ ( {θ}_{thigh} \geq {θ}_{thigh}^{THS} + 5 ‖ {θ}_{thigh} < 0 )\end{matrix} & ( 7 )\end{matrix} \\$$

The target knee angle (θₖₙₑₑ^{des}) and the target ankle angle (θₐₙₖₗₑ^{des}) discussed above may be determined/utilized while the controller is in the Lifting state, as noted above (e.g., when the ground reaction force GRF is lower than a predefined threshold). In some implementations, when the ground reaction force (GRF) is higher than a fixed threshold (GRF^{THS}) the prosthesis controller transitions from the Lifting state to the Standing state. In some instances, the desired knee torque (or target knee torque) is defined in Standing as a continuous function of the knee position, imitating the quasi-stiffness shape of the intact biological leg. However, in some instances, the desired or target torque-angle relationship is not fixed, but changes as a function of the of the knee position when the controller switches from Lifting to Standing (θₖₙₑₑ⁰) (e.g., the knee position measured at the transition from Lifting to Standing). Such torque modulation can be based on a heuristic algorithm inspired by non-amputee biomechanics.

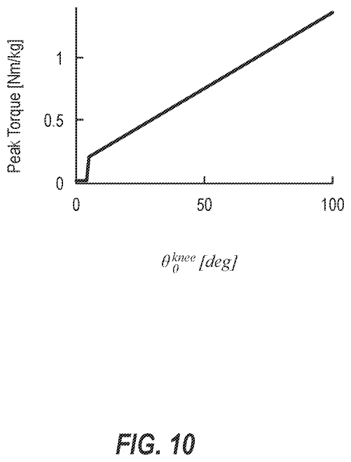

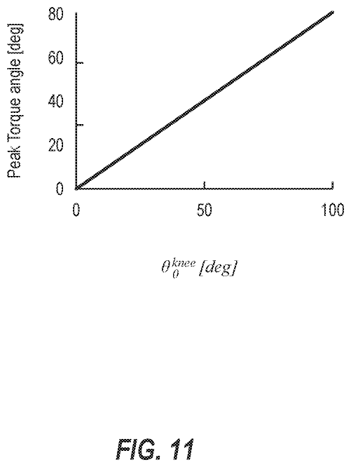

FIG. 10 illustrates an example relationship between knee position at the start of Standing with respect to peak knee torque. As shown in FIG. 10, the peak knee torque changes as a function of the measured knee angle at the transition between Lifting and Standing (θₖₙₑₑ⁰), which may be regarded as an indicator of the step height and can be determined by a Lifting controller. In some implementations, the knee angle at which the peak knee torque is generated (θₖₙₑₑ^{T}^{ₘₐₓ}) also changes with the measured knee angle at the transition between Lifting and Standing (θₖₙₑₑ⁰) as shown in FIG. 11.

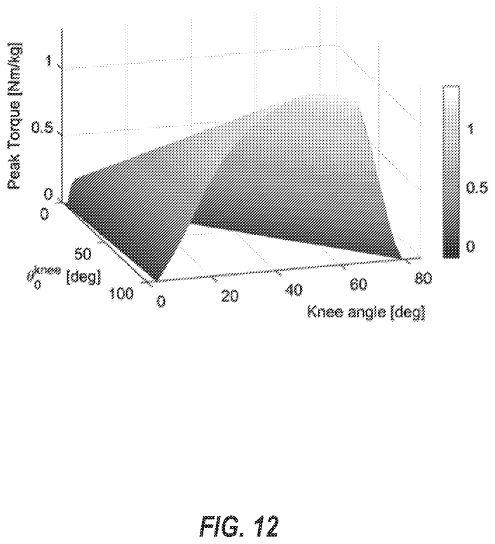

The desired torque may then (e.g., during Standing) be encoded in the controller using a bi-dimensional look-up table, which may improve computational efficiency. FIG. 12 illustrates desired torque as a function of (i) the measured knee angle at the transition between Lifting and Standing (θₖₙₑₑ⁰) and (ii) the currently measured knee angle. An impedance controller may be used if the measured knee angle exceeds the knee angle at the transition between Lifting and Standing (θₖₙₑₑ⁰) Such an impedance controller may respond to any movements of the knee joint in the flexion direction, thereby providing additional stability in Standing.

Thus, in some implementations, with the disclosed adaptive controller, larger knee extension torque can be produced when the powered prosthesis transitions between Lifting and Standing at a larger knee flexion angle, ultimately injecting higher mechanical energy into the stair-climbing cycle. Moreover, if the powered prosthesis transitions between Lifting and Standing with the knee fully extended, for example when the user shuffles around with no intention to climb a step, the desired torque can be defined solely by the impedance component, which may stabilize the knee joint and prevent it from collapsing under the user's body weight. Thus, the disclosed Standing controller may adapt the desired knee torque and energy injection with the step height while providing the user with the freedom to take the step at their preferred cadence.

In some implementations, the ankle behavior during Standing is defined using an impedance controller with an adaptive virtual equilibrium angle (θₐₙₖₗₑ^{EQ}). Due to the adaptive nature of the Lifting controller discussed above, the angle of the powered ankle joint at the transition between Lifting and Standing is not fixed, but changes as a function of the user's residual limb orientation and acceleration (e.g., as defined by Equation (6) and Equation (7)). Thus, at the transition between Standing and Lifting, the equilibrium angle of the ankle (θₐₙₖₗₑ^{EQ}) may be set to the measured ankle angle. Then (e.g., during Standing), the equilibrium angle of the ankle may change linearly with the knee position as defined by Equation (8).

$${θ}_{ankle}^{EQ} = \frac{{θ}_{knee}^{0}}{{θ}_{ankle}^{0}} ( {θ}_{knee}^{meas} - {θ}_{knee}^{0} ) \qquad ( 8 )$$

Accordingly, with the disclosed Standing controller, the powered ankle joint may move from whatever its initial angle is when the prosthetic foot contacts the step to a neutral position (i.e., 0°) when the powered knee joint is fully extended. Thus, the powered ankle can contribute positive power to the Standing movement. At the same time, if the subject shuffles around without taking a step, the ankle may stay in a neutral position while providing compliant support to help the user balance while standing.

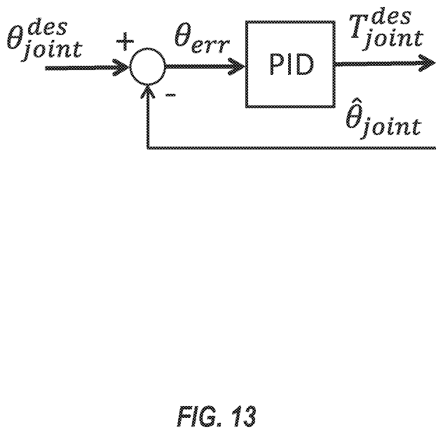

The desired torque(s) and/or angle(s) defined by the Standing and Lifting controllers as discussed above (e.g., the target knee angle, the target ankle angle, the target knee torque, the target ankle equilibrium angle, etc.) may be enforced by one or more dedicated low-level controllers using a hybrid feedforward/feedback approach. By way of non-limiting example, during Lifting, closed-loop position controllers (as shown in FIG. 13) may be used to impose the desired joint angles at the ankle and knee joints. As shown in FIG. 13, for each powered joint, the closed-loop position controller may take as input the desired angle (θⱼₒᵢₙₜ^{des}) and compare it to the measured angle (\hat{θ}ⱼₒᵢₙₜ), which may be estimated using a complementary filter. The angle error (θₑᵣᵣ) may be fed to a PID controller that determines the desired torque command (Tⱼₒᵢₙₜ^{des}).

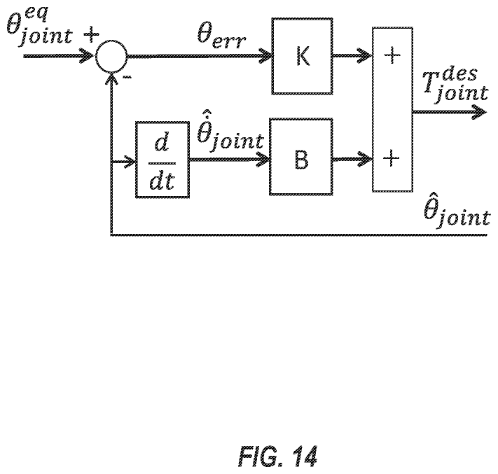

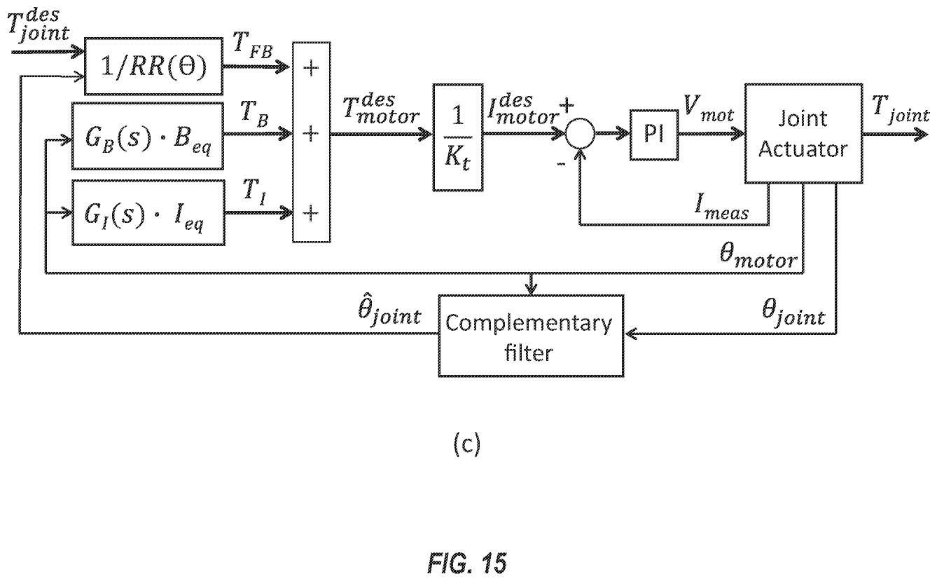

In Standing, the ankle joint uses a virtual impedance controller (as shown in FIG. 14) with predefined stiffness and damping parameters (K, B) to define the desired torque command (Tⱼₒᵢₙₜ^{des}). The desired torque command is then, in some instances, fed to a low-level torque controller (FIG. 15). As shown in FIG. 15, the low-level torque controller comprises a feedforward command based on the position-dependent transmission ratio (RR (θ)). In addition, two compensators are used to reduce the apparent impedance (i.e., viscosity and inertia) of the transmission system (e.g., to improve the fidelity of the virtual impedance controller). The first compensator (i.e., G_{B}(s)·B_{eq}) takes as input the motor position (θₘₒₜₒᵣ) and generates an online estimate of the viscous torque (T_{B}) due to the linear actuator. The second compensator (i.e., G_{I}(s)·I_{eq}) takes as input the motor position (θₘₒₜₒᵣ) and computes a scaled and low-pass filtered estimate of the transmission inertia (T₁).

Although the present disclosure touches on functions that may be associated with a Lifting controller and functions that may be associated with a Standing controller, one will appreciate, in view of the present disclosure, that the functions associated with the different states discussed herein may be performed by the same controller and/or otherwise logically divided among any number of processing/computing devices/components.

### Example Methods

The following discussion now refers to a number of methods and method acts that may be performed in accordance with the present disclosure. Although the method acts are discussed in a certain order and illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein.

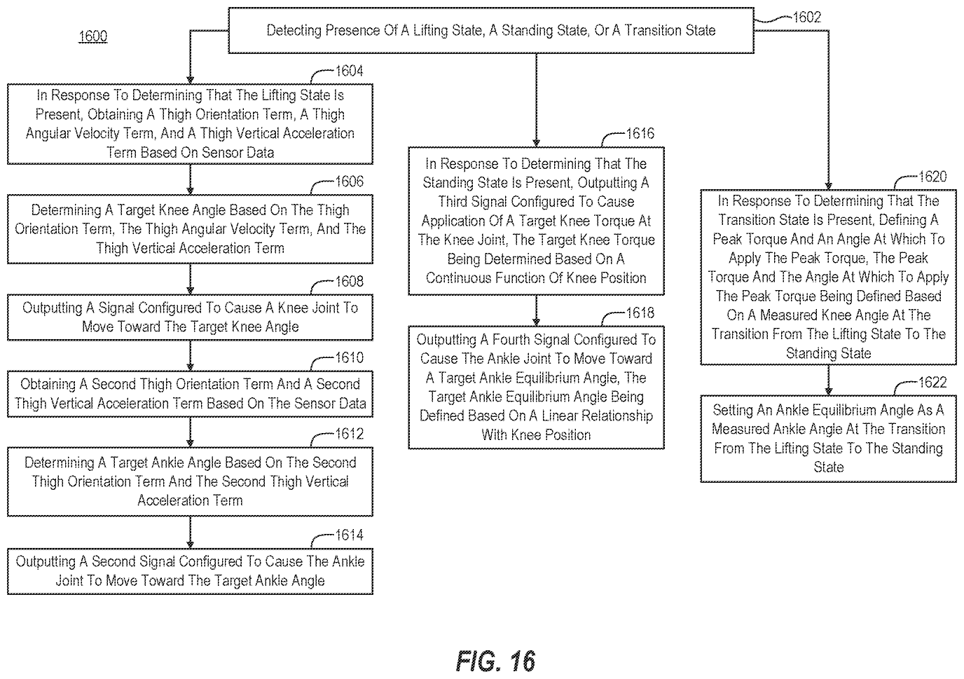

FIG. 16 illustrates an example flow diagram 1600 depicting acts associated with adaptively controlling powered joint movement during climbing tasks. The acts depicted in flow diagram 1600 may be performed utilizing various hardware elements discussed hereinabove, such as controllers (e.g., of control unit and battery 225), sensor(s), motors, etc. A controller may comprise one or more processing devices and may comprise or access one or more hardware storage devices to facilitate execution of stored instructions to carry out one or more of the acts/functions described herein.

Act 1602 of flow diagram 1600 includes detecting presence of a lifting state, a standing state, or a transition state. The state determined to be present may be based on a detected ground reaction force (GRF). For example, when the GRF is below a predefined GRF threshold, the lifting state may be determined to be present. In contrast, when the GRF is above the predefined GRF threshold, the standing state may be determined to be present. The transition state may comprise a transition between the lifting state and the standing state.

Flow diagram 1600 illustrates various acts performed in response to determining that the lifting state is present, including acts 1604, 1606, 1608, 1610, 1612, and 1614.

Act 1604 includes, in response to determining that the lifting state is present, obtaining a thigh orientation term, a thigh angular velocity term, and a thigh vertical acceleration term based on sensor data. The sensor data may be obtained utilizing one or more sensors configured to sense attributes of a residual limb of an above-knee amputee. In some instances, the thigh orientation term is proportional to an orientation of a user thigh with respect to gravity when a first thigh orientation threshold is satisfied, and the thigh orientation term may be set to zero when the first thigh orientation threshold is not satisfied (e.g., according to Equation (1) discussed above).

In some instances, the thigh angular velocity term is proportional to a positive angular velocity of a user thigh (e.g., the thigh vertical acceleration term may depend upon a vertical acceleration of a user thigh with respect to gravity) (e.g., according to Equation (2)).

In some implementations, the thigh vertical acceleration term is determined by determining a double integral of a first quantity and multiplying the double integral by a non-constant factor (e.g., according to Equation (3) and Equation (4)). For example, the first quantity may comprise a first factor subtracted from the vertical acceleration of the user thigh with respect to gravity, and the non-constant factor may change as a function of thigh orientation. The non-constant factor may be constant for thigh orientations below a second thigh orientation threshold, and, for thigh orientations that exceed the second thigh orientation threshold, the non-constant factor may be defined by a decreasing linear relationship that decreases linearly until reaching zero at a predetermined offset from the second thigh orientation threshold. In some instances, the second thigh orientation threshold associated with the thigh vertical acceleration term is the same as the thigh orientation threshold associated with the thigh orientation term.

Act 1606 includes determining a target knee angle based on the thigh orientation term, the thigh angular velocity term, and the thigh vertical acceleration term. The target knee angle may comprise a summation of the thigh orientation term, the thigh angular velocity term, and the thigh vertical acceleration term.

Act 1608 includes outputting a signal configured to cause a knee joint to move toward the target knee angle. In some instances, the target knee angle is enforced utilizing one or more dedicated low-level controllers that utilize a hybrid feedforward/feedback approach (e.g., as shown and described with reference to FIGS. 13 through 15).

Act 1610 includes obtaining a second thigh orientation term and a second thigh vertical acceleration term based on the sensor data. The second thigh orientation term may be determined in accordance with Equation (5), as discussed hereinabove. For example, in some implementations, the second thigh orientation term is zero for user thigh orientation angles lower than zero. Furthermore, in some instances, the second thigh orientation term is proportional to thigh orientation angle when the thigh orientation angle is within a first range of thigh orientation angles. Still furthermore, in some instances, the second thigh orientation term is defined by a decreasing linear relationship to approach a shank angle when the thigh orientation angle is within a second range of thigh orientation angles. The second range of thigh orientation angles is greater than the first range of thigh orientation angles. Furthermore, in some instances, the second thigh orientation term is equal to the shank angle when the thigh orientation angle is greater than the second range of thigh orientation angles.

The second thigh vertical acceleration term may be determined in accordance with Equation (6) and Equation (7), as discussed hereinabove. For example, the second thigh vertical acceleration term may depend upon a vertical acceleration of a user thigh with respect to gravity. The second thigh vertical acceleration term may be determined by determining a second double integral of a second quantity and multiplying the double integral by a second non-constant factor. The second quantity may comprise a second factor subtracted from the vertical acceleration of the user thigh with respect to gravity. The second non-constant factor may change as a function of thigh orientation. For example, the second non-constant factor may be constant for thigh orientations below a third thigh orientation threshold, and, for thigh orientations that exceed the third thigh orientation threshold, the second non-constant factor may be defined by a decreasing linear relationship that decreases linearly until reaching zero at a second predetermined offset from the third thigh orientation threshold. In some instances, the third thigh orientation threshold is the same as the thigh orientation threshold and/or the second thigh orientation threshold discussed hereinabove with reference to the thigh orientation term and/or the thigh vertical acceleration term, respectively. Furthermore, in some instances, the second predetermined offset may be the same as the predetermined offset referred to above in association with the thigh vertical acceleration term.

Act 1612 includes determining a target ankle angle based on the second thigh orientation term and the second thigh vertical acceleration term. In some instances, the target ankle angle comprises a summation of the second thigh orientation term and the second thigh vertical acceleration term.

Act 1614 includes outputting a second signal configured to cause the ankle joint to move toward the target ankle angle. In some instances, the target ankle angle is enforced utilizing one or more dedicated low-level controllers that utilize a hybrid feedforward/feedback approach (e.g., as shown and described with reference to FIGS. 13 through 15).

Flow diagram 1600 illustrates various acts performed in response to determining that the standing state is present, including acts 1616 and 1618.

Act 1616 includes, in response to determining that the standing state is present, outputting a third signal configured to cause application of a target knee torque at the knee joint, the target knee torque being determined based on a continuous function of knee position. For example, the target knee torque may be determined as a function of (i) a measured knee angle at a transition between a Lifting state and a Standing state and (ii) a currently measured knee angle (e.g., as shown and described hereinabove with reference to FIG. 12).

Act 1618 includes outputting a fourth signal configured to cause the ankle joint to move toward a target ankle equilibrium angle, the target ankle equilibrium angle being defined based on a linear relationship with knee position. The target ankle equilibrium angle may be determined in accordance with Equation (8) discussed hereinabove.

Flow diagram 1600 illustrates various acts performed in response to determining that the lifting state is present, including acts 1620, 1622.

Act 1620 includes, in response to determining that the transition state is present, defining a peak torque and an angle at which to apply the peak torque, the peak torque and the angle at which to apply the peak torque being defined based on a measured knee angle at the transition from the lifting state to the standing state. For example, the peak torque and the angle at which to apply the peak torque may be determined based on linear relationships with the measured knee angle at the transition from the lifting state to the standing state (e.g., as shown and described hereinabove with reference to FIGS. 10 and 11.

Act 1622 includes setting an ankle equilibrium angle as a measured ankle angle at the transition from the lifting state to the standing state.

In some implementations, by implementing one or more of the acts associated with flow diagram 1600, a powered knee and ankle prosthesis controller may adaptively update a target knee angle, a target ankle angle, a target knee torque, and/or a target ankle equilibrium angle based on updated sensor data, thereby enabling the controller to adapt to variable stair height, user cadences, and/or user gait patterns that may be encountered in real-world scenarios.

### Examples

It shall be noted that these experiments and results are provided by way of illustration and were performed under specific conditions using a specific embodiment or embodiments. Aspects of the experimental protocol(s) discussed below may be applied in real-world and/or end-use contexts. However, neither these experiments (including the specific experimental conditions or embodiment(s)) nor their results shall be used to limit the scope of the present disclosure.

Participant Information

One subject with above-knee amputation participated in these experiments. The subject was 27 years old, weighed 65 kg, was a 1.7 m tall male, and had an above knee amputation for 6 years at the time of the experiments. The subject had experience with the powered prosthesis used in the experiments (i.e., the Utah Lightweight Leg, which corresponds to the powered knee and ankle prosthesis 200 discussed hereinabove with reference to FIG. 2), but did not have experience with the adaptive controller utilized.

Experimental Protocol

The experiment preparation took place before data collection. The subject donned the Utah Lightweight Leg (e.g., the powered knee and ankle prosthesis 200 discussed above). A certified prosthetist adjusted the build height of the prosthesis using the standard pylon and ensured proper alignment of the knee and ankle joints. After the prosthesis fitting was completed, the subject donned an IMU-based motion capture system (e.g., MTw Awinda, Xsens). Eight sensors were placed on the subject. Two sensors were placed on the top of each foot, two on each shank just below the knee joint, two on the outside of each thigh, one in the center of the lower back, and one sensor on the sternum. Then, the motion capture system was calibrated to the subject. The calibration protocol consisted of having the subject stand still for 5 seconds, take 3 strides forward, turn around, take another 3 strides, and return the original standing position. After the system calibration, the subject practiced climbing stairs with the disclosed controller for about 15 minutes on both the 4 inch and 7 inch staircases. During practice, the controller parameters were fine-tuned by the experimenter based on the subject's preference. The whole experiment preparation lasted about 30 minutes.

Although the disclosed controller relies, in some implementations, on a series of bioinspired curves (FIGS. 3 through 12), modulated using several coefficients and parameters, only four parameters were actually tuned during practice (i.e., k₁^{knee}, k₁^{ankle}, k₄^{0,knee}, k₄^{0,ankle}). All the other control gains and parameters were kept constant at values determined offline from the analysis of nonamputee and amputee biomechanics. To fine tune the controller, the subject was first asked to place the prosthesis on the step in front of them without climbing it. With the prosthesis on the step, k₁^{knee} and k₁^{ankle} were fine-tuned to achieve a natural, comfortable posture, while making sure that the prosthesis shank was slightly tilted forward, and the prosthetic foot was flat on the step. Then, the subject was asked to climb the staircase step-by-step, leading with the sound side. As the subject climbed the steps, k₄^{0,knee} and k₄^{0,ankle} were fine-tuned, making sure that the powered prosthesis cleared the steps. Finally, the subject was asked to climb stairs step-over-step and verified the disclosed controller. The whole tuning procedure was performed on the 7-in staircase.

After the experiment preparation was completed, the subject performed the experimental protocol for data collection. The subject ascended two staircases of 4 steps, each with 3 different gait patterns. The first staircase is the maximum ADA compliant step height of 7 inches (18 cm), the second staircase is the minimum ADA compliant step height of 4 inches (10 cm). First, the subject used the step-over-step gait pattern, which is the most common way to climb stairs for non-amputee individuals. When climbing stairs step-over-step, each foot is placed one step above the other foot. Then, the subject used a step-by-step gait pattern, which is the most common stair ascent method for above-knee amputees using conventional prostheses. When climbing stairs step-by-step, the leading foot is placed one step above, and the following foot is brought to match on the step of the leading foot. Finally, the subject used a two-steps gait pattern, which is less common and mostly used when in a hurry. When climbing stairs with the two-steps gait pattern, the leading leg is taking two-steps at a time and the following leg is brought to match that step. The subject performed 5 ascents for each gait pattern and staircase. The subject climbed the staircase at their preferred cadence. Thus, the protocol tested several combinations of gait patterns and stair heights, while leaving the gait cadence up to the user's preference.

Data acquired from the motion capture systems and the sensors embedded in the powered prosthesis were processed offline. The motion capture system provided the kinematics of the ankle, knee, and hip joint, the orientation of the leg segments and the Cartesian-space position of the toe, ankle, knee, and hip joints, for both the prosthesis side and the sound side. The powered prosthesis provided the kinetics and kinematics of the prosthetic ankle and knee joints. Data recorded from the motion capture system and the powered prosthesis were synchronized online through Wi-Fi. The synchronized raw data was filtered offline using a zero-lag low-pass Butterworth filter with a cutoff frequency of 8 Hz. Joint angular velocities, accelerations, and power were calculated post filtering. Segmentation indexes for stance and swing phase during stair ascent were determined using the gait state parameters defined online by the powered prosthesis controller. Full strides started and ended at toe off on the prosthesis side. After segmentation, each stride was resampled to 1000 samples, and the time was normalized as percent of stride accomplishment. Energy injection was calculated as the integral of the joint torque-angle curve, which is theoretically equivalent to integrating mechanical power over time but does not require offline calculation of the joint velocity by numerical differentiation, which is typically noisy and involves filtering. Moreover, energy injection was calculated for stance phase only to isolate the ability of the disclosed Stance controller to adapt the energy injection to both the step height (i.e., 4 inch vs. 7 inch) and the gait pattern (e.g., Two-Steps vs. Step-over-Step).

Experimental Results

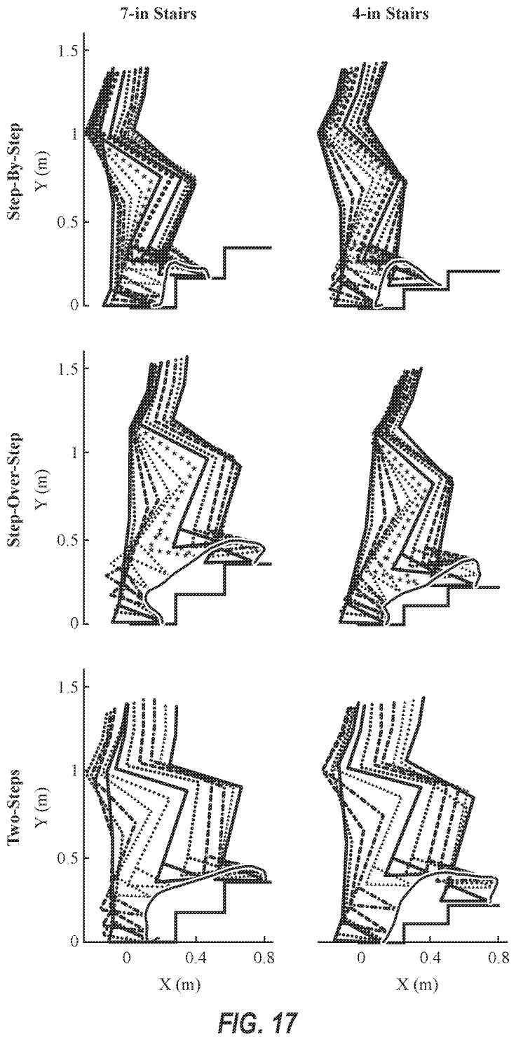

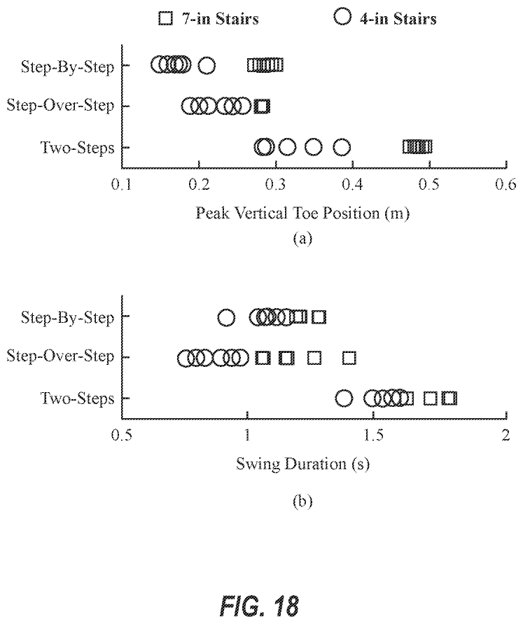

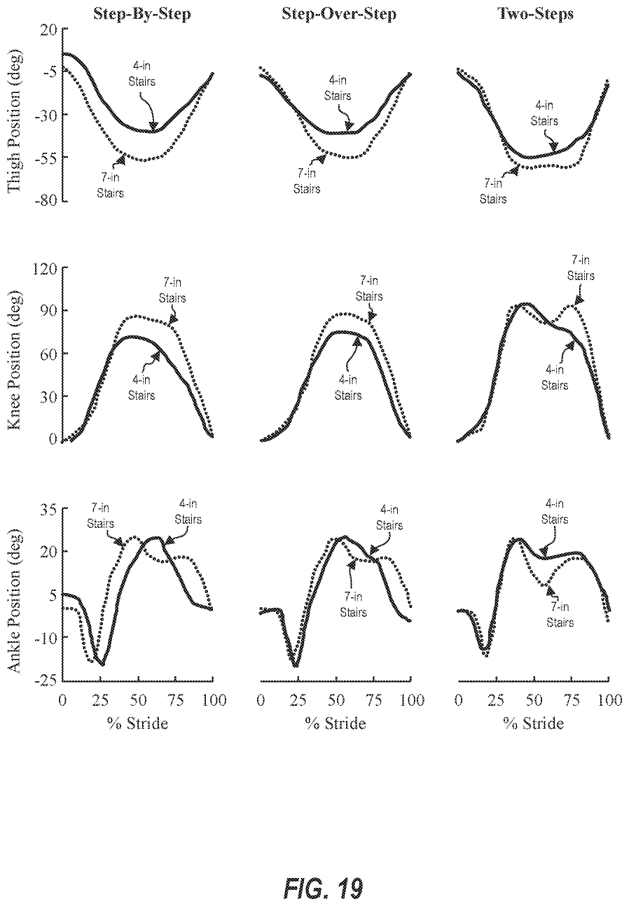

The swing trajectories for different stair heights and gait patterns were visibly different, as is evident in FIG. 17, which illustrates the swing trajectory of the powered prosthesis from cartesian space for the different stair heights and gait patterns. Differences are also evident in FIG. 18, which shows the duration of swing for each of the different conditions (as well as peak vertical toe position for each of the different conditions). The maximum knee angle during swing was 88.5±2.9°, 88.3±2.6°, and 96.0±1.9° for step-by-step, step-over-step and two-steps gait patterns for the 7-in stairs, respectively (see FIG. 19, which illustrates kinematic analysis of the thigh segment, knee joint, and ankle joint for different gait patterns and stair heights). The maximum knee angle during swing was 74.9±2.8°, 73.6±1.8°, and 95.9±1.5° for step-by-step, step-over-step and two-steps gait patterns for the 4-in stairs, respectively (see FIG. 19). Thus, there were substantial differences in the maximum knee flexion angle between two-steps gait pattern and the step-by-step and step-over-step gait patterns but almost no difference between the step-by-step and step-over-step gait patterns. As expected, a noticeable difference existed between stair heights, with an 18% and 20% increase in knee flexion between the 7-in and 4-in stairs for the step-by-step and step-over-step gait patterns, respectively. Thus, the disclosed controller provided sufficient foot clearance and proper foot placement for all observed gait patterns and stair heights.

The swing duration was calculated from the moment the prosthetic foot left the ground to the moment the prosthetic foot touched the ground, as determined by the finite-state machine. Because the powered prosthesis continuously follows the residual-limb movements, the swing duration reflects the user's self-selected cadence. The swing duration ranged from 0.76 s for the 4-in stairs with step-over-step gait pattern and 1.80 s for the 7-in stairs with the two-steps gait pattern. The step-over-step gait pattern on the 7-in stairs had the highest deviation in swing duration, with a minimum of 1.1 seconds and a maximum of 1.4 seconds (see FIG. 18). The disclosed controller enabled the subject to change his cadence when climbing stairs with different heights or using different gait patterns.

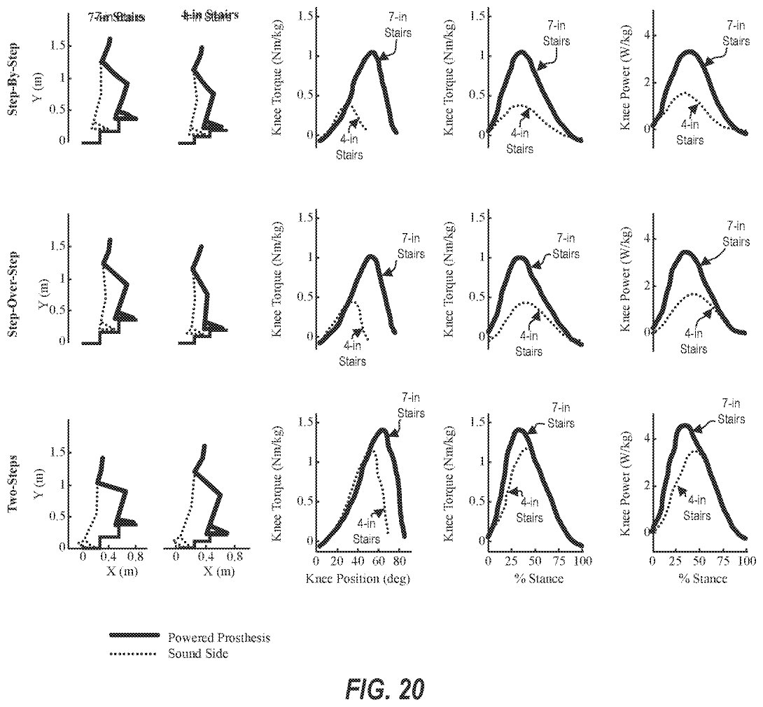

The prosthesis angle at the start of stance varied for different stair heights and gait patterns (see FIGS. 20, which illustrates kinematic analysis of a standing phase for different gait patterns and stair heights). The knee angle at the start of stance was 75.3±1.0°, 74.5±1.9°, and 84.4±5.3° for step-by-step, step-over-step and two-steps gait patterns for the 7-in stairs, respectively. The knee angle at the start of stance was 51%, 49%, and 23% larger on the 7-in stairs compared to the 4-in stairs for the step-by-step, step-over-step, and two-steps gait pattern, respectively. Also, the knee angle at the start of stance was 50.0±6.5°, 49.9±3.4°, and 68.8±3.6° for step-by-step, step-over-step and two-steps gait patterns for the 4-in stairs, respectively. Thus, the knee angle was 13% and 38% larger for the two-steps gait pattern compared to the single-step gait patterns for the 7-in and 4-in stair heights, respectively. The disclosed controller changed the prosthesis knee angle at the start of stance adapting to the different gait pattern and stair heights.

The peak of the prosthesis knee torque changed with different stair heights and gait patterns (see FIG. 20). The peak knee torque was 1.06±0.06 Nm/kg, 1.03±0.04 Nm/kg, and 1.44±0.15 Nm/kg for step-by-step, step-over-step and two-steps gait patterns for the 7-in stairs, respectively. Thus, the peak knee torque increased by 38% and 166% for the two-steps gait pattern compared to the single-step gait patterns for the 7-in and 4-in stairs, respectively. The peak knee torque was 0.40±0.15 Nm/kg, 0.50±0.10 Nm/kg, and 1.20±0.08 Nm/kg for step-by-step, step-over-step and two step gait patterns for the 4-in stairs, respectively (as shown in FIG. 20). Thus, the peak knee torque measured for the 7-in stairs was 164%, 106% and 20% larger compared to the 4-in stairs for the step-by-step, step-over-step, and two-steps gait pattern, respectively.

The timing of the prosthesis knee torque peak varied for different stair heights and gait patterns (see FIG. 20). The peak knee torque was provided at a knee angle of 29.9, 33.6, and 52.7° for the step-by-step, step-over-step, and two-steps gait pattern for the 4-in stairs and 53.6, 52.6, and 64.2° for the step-by-step, step-over-step, and two-steps gait pattern for the 7-in stairs. Thus, the knee angle at peak torque was 30% smaller than the knee angle at the start of stance. The disclosed controller changed the torque based on the gait pattern and stair height.

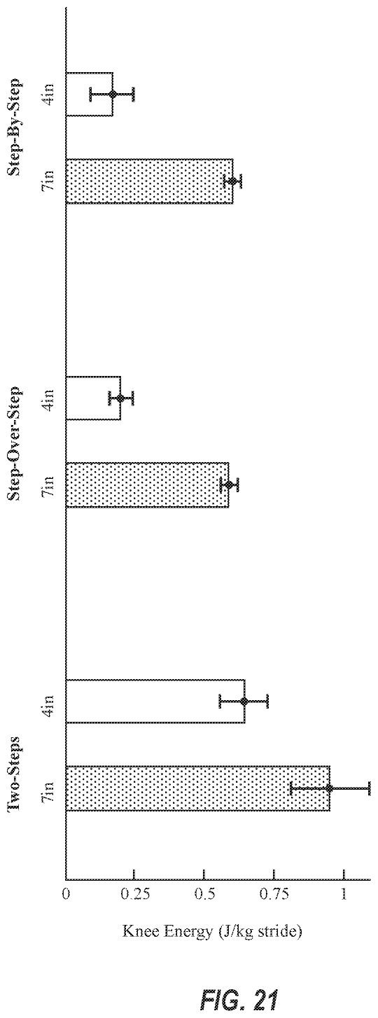

Joint power and energy injection for different stair heights and gait patterns were assessed (see FIG. 21, which illustrates further kinematic analysis of a standing phase for different gait patterns and stair heights). The peak knee power was 3.37±0.25 W/kg, 3.39±0.27 W/kg, and 4.71±0.20 W/kg for the step-by-step, step-over-step, and two-steps gait patterns for the 7-in stairs, respectively. The peak knee power was 1.52±0.63 W/kg, 1.90±0.43 W/kg, and 3.60±0.68 W/kg for the step-by-step, step-over-step, and two-steps gait patterns for the 4-in stairs, respectively. The energy injected in stance was 0.60±0.03 J/kg, 0.59±0.03 J/kg, and 0.95±0.14 J/kg for the step-by-step, step-over step and two-step gait patterns for the 7-in stairs, respectively. The energy during stance was 0.17±0.08 J/kg, 0.20±0.04 J/kg, and 0.64±0.08 J/kg for the step-by-step, step-over-step, and two-steps gait patterns for the 4-in stairs, respectively. The two-steps gait pattern injected 60% and 246% more energy compared to the single step gait patterns for the 7-in and 4-in stairs, respectively. The 7-in stairs injected 253%, 195%, and 48% more energy compared to the 4-in stairs for the step-by-step, step-over-step, and two-steps gait patterns, respectively (as shown in FIG. 21). The disclosed controller injected a different amount of energy into the gait cycle depending on the gait pattern and stair height.

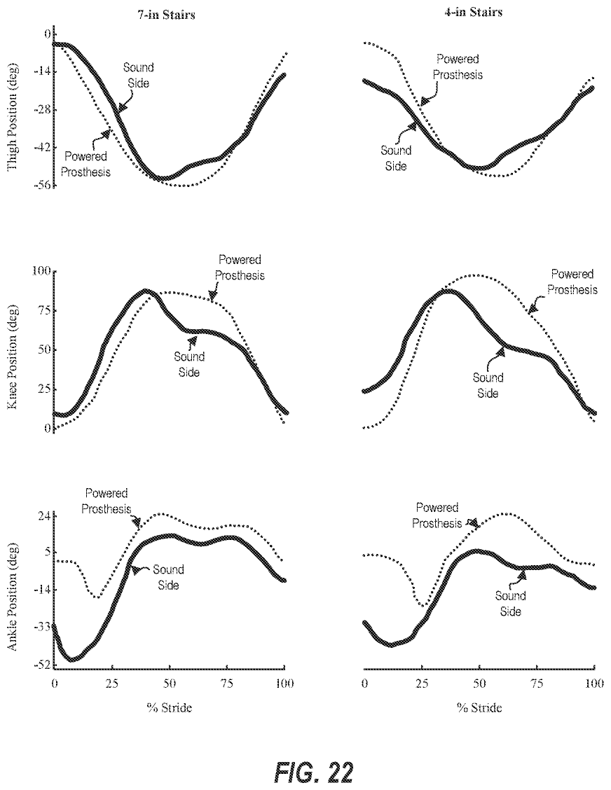

A kinematic analysis was performed between the sound side and the prosthesis side for the thigh orientation, knee angle and ankle angle, as shown in FIG. 22. The peak knee angle of the knee on the sound side was 88.5±2.9° and 70.1±3.7° for the 7-in and 4-in stairs, respectively. At toe off, or 0% stride, the sound side for the 4-in stairs experiences some initial flexion of −8.0±2.2° for the thigh and 17.1±2.5° for the knee compared to the sound side for the 7-in stairs and both stair heights of the prosthesis side where the knee and thigh position at toe off are closer to a neutral position (see FIG. 22). Additionally, the ankle on the sound side experiences −32.1±10.8° and −27.6±11.0° of plantarflexion at toe off for the 7-in and 4-in stair height, respectively, where the prosthesis side starts with a more neutral ankle position for both the 7-in and 4-in stair height, respectively. The peak plantarflexion angle on the prosthesis side was −25.2±0.07° and −25.2±0.07° for the 7-in and 4-in stairs, respectively. The peak plantarflexion angle on the sound side was −51.4±2.3° and −39.7±4.7° for the 7-in and 4-in stairs, respectively. Thus, the sound side experiences 104% and 58% more plantarflexion compared to the prosthesis side for the 7-in and 4-in stair height, respectively. The peak dorsiflexion angle on the prosthesis side was 20.0±1.0° and 20.2±1.7° for the 7-in and 4-in stairs, respectively. The peak dorsiflexion angle on the sound side was 14.8±1.5° and 7.8±2.2° for the 7-in and 4-in stairs, respectively. The disclosed controller enabled the subject to climb stairs with different heights despite noticeable differences between the kinematics of the sounds side and the prosthesis side.

Ascending stairs in the real world requires controllers that synchronize the movements of the powered prosthetic joints with the movements of the user's residual limb. If the controller moves too fast or too slow with respect to the user's residual limb, then the prosthesis will hit the stairs, causing the user to trip and fall. Available stair controllers for powered prostheses cannot synchronize with the user. Therefore, users must learn how to time their residual limb movements with the prosthesis to ensure that the step is cleared. Because the swing time is fixed, changing cadence is not possible with available stair ascent controllers. In contrast, the disclosed adaptive Swing controller (see FIGS. 3 through 9) enables climbing stairs at a variable cadence (from 0.76 s/stride to 1.8 s/stride), which may enable ambulation on staircases with different heights (4-in., 7-in.) or using different gait patterns (step-by-step, step-over-step, two-steps). The experimental results included herein indicate that the disclosed Swing controller enables climbing stairs with different heights and gait patterns by intrinsically synchronizing with the user's thigh movements.

Adaptation to different staircases or gait patterns requires the position of the prosthetic foot at the end of swing to match the stair height. If the prosthetic knee is too flexed, then the prosthetic foot hovers above the step. If the prosthetic knee is not flexed enough, the prosthetic foot does not clear the last step. Moreover, the angle of the prosthetic joints at the start of stance is important. The knee joint should be flexed to an extent that ensures the prosthesis shank orientation is past the vertical line defined by gravity so that the user's center of mass is above the prosthesis. The ankle should be dorsiflexed to ensure the prosthetic foot stays flat on the step. Available stair controllers are tuned for a specific staircase and gait pattern so that proper foot placement is achieved. Outside of the specific tuning conditions, these controllers cannot provide proper toe clearance and foot placement. In contrast, the disclosed adaptive Swing controller can achieve a suitable prosthesis orientation for all tested stair heights and gait patterns by changing the knee flexion continuously with the thigh angle (see FIGS. 17-18). Similarly, the experimental results included herein indicate that the ankle angle is continuously adapted based on gravity, enabling the prosthetic foot to remain perpendicular to the step for all tested stair heights and gait patterns. Thus, the experimental results included herein indicate that the disclosed adaptive Swing controller provides proper foot placement for different stair heights and gait patterns.

To facilitate sufficient toe clearance, in the disclosed controller, the prosthesis joint angles depend on both the thigh angle, velocity and vertical acceleration as defined by Equations (1) through (7). In general, the velocity dependency, Equation (2), appears to help clearing the intermediate step, whereas the vertical acceleration term, Equation (3) and Equation (4), appears to have a major impact in clearing the first step, when the residual limb is not rotating (FIG. 17-18). Thus, the experimental results indicate that the residual limb orientation, velocity, and vertical acceleration are suitable combination of inputs to continuously adapt the prosthesis trajectory during stair ascent.

Climbing stairs with different stair heights or gait patterns requires different torque generation and mechanical energy injection. However, available stair ascent controllers use either a fixed, pre-programmed stance torque profile or joint impedance. Therefore, they cannot change torque generation or energy injection. To address this limitation, the disclosed Stance controller automatically increases the maximum knee torque proportionally to the knee flexion angle at the beginning of stance (FIGS. 10-12). The experimental results indicate that because the knee flexion angle at the beginning of stance is proportional to the stair height (FIGS. 10-12, 20-21), the energy injected by the prosthesis is also proportional to the stair height (FIGS. 20-21). Thus, the experimental results indicate that the disclosed Stance controller provides sufficient modulation of torque and energy injection to enable climbing stairs with different heights and gait patterns.

Inspired by biological knee behavior, the disclosed controller sets the knee angle at which the peak knee torque is provided proportional to the knee range of motion (FIG. 10-12). The experimental results indicate that the torque-angle relationship is scaled linearly on the knee range of movement (FIG. 20-21), and the knee angle at peak torque changes depending on the stair height and gait pattern (FIG. 20-21). Because the energy injection is independent with respect to time, the user was able to climb stairs at their desired cadence while still receiving the assistance needed. Thus, the experimental results indicate that the disclosed Stance controller synchronizes energy injection to the user's movements when climbing stairs with different heights and gait patterns.

In the disclosed Stance controller, the ankle movements are synchronized to the knee movements, using a dedicated adaptive function (Equation 8). The experimental results show that different ankle angles are achieved at the beginning of stance for different stair heights and gait patterns (FIGS. 20-21). However, for all tested conditions the ankle angle gradually returns to neutral as the knee extends (FIGS. 20-21). Thus, the experimental results indicate that the disclosed Stance controller indirectly synchronizes the ankle movements to the residual limb movements when climbing stairs with different heights and gait patterns.

In some embodiments, the disclosed controller advantageously uses a finite-state machine (FIGS. 13-15) with only two states (Stance and Swing), whereas other stair controllers use at least four states. In general, reducing the number of states in the finite-state machine reduces the probability of a wrong transition being triggered, improving robustness. Moreover, reducing the number of states reduces the number of control parameters that need to be tuned, reducing tuning time.

### Additional Example Aspects

Embodiments of the present disclosure may include, but are not necessarily limited to, features recited in the following clauses:

Clause 1: a powered prosthesis configured to adaptively control powered joint movement during climbing tasks, the prosthesis comprising: a knee joint; one or more sensors configured to capture sensor data associated with a residual limb of a user; a controller comprising one or more processors and one or more hardware storage devices storing instructions that are executable by the one or more processors to configure the controller to: obtain a thigh orientation term, a thigh angular velocity term, and a thigh vertical acceleration term based on the sensor data; determine a target knee angle based on the thigh orientation term, the thigh angular velocity term, and the thigh vertical acceleration term; and output a signal configured to cause the knee joint to move toward the target knee angle.

Clause 2: the powered prosthesis of Clause 1, wherein the instructions are executable by the one or more processors to configure the controller to adaptively update the target knee angle based on updated sensor data, thereby enabling the controller to adapt to variable stair height, user cadences, and/or user gait patterns.

Clause 3: the powered prosthesis of Clause 1 or Clause 2, wherein the thigh orientation term is proportional to an orientation of a user thigh with respect to gravity when a first thigh orientation threshold is satisfied.

Clause 4: the powered prosthesis of Clause 3, wherein the thigh orientation term is set to zero when the first thigh orientation threshold is not satisfied.

Clause 5: the powered prosthesis of any one of Clauses 1 through 4, wherein the thigh angular velocity term is proportional to a positive angular velocity of a user thigh.

Clause 6: the powered prosthesis of any one of Clauses 1 through 5, wherein the thigh vertical acceleration term depends upon a vertical acceleration of a user thigh with respect to gravity.

Clause 7: the powered prosthesis of Clause 6, wherein the thigh vertical acceleration term is determined by: determining a double integral of a first quantity, the first quantity comprising a first factor subtracted from the vertical acceleration of the user thigh with respect to gravity; and multiplying the double integral by a non-constant factor.

Clause 8: the powered prosthesis of Clause 7, wherein the non-constant factor changes as a function of thigh orientation.

Clause 9: the powered prosthesis of Clause 8, wherein the non-constant factor is constant for thigh orientations below a second thigh orientation threshold, and wherein, for thigh orientations that exceed the second thigh orientation threshold, the non-constant factor is defined by a decreasing linear relationship that decreases linearly until reaching zero at a predetermined offset from the second thigh orientation threshold.

Clause 10: the powered prosthesis of any one of Clauses 1 through 9, further comprising an ankle joint.

Clause 11: the powered prosthesis of Clause 10, wherein the instructions are executable by the one or more processors to configure the controller to: obtain a second thigh orientation term and a second thigh vertical acceleration term based on the sensor data; determine a target ankle angle based on the second thigh orientation term and the second thigh vertical acceleration term; and output a second signal configured to cause the ankle joint to move toward the target ankle angle.

Clause 12: the powered prosthesis of Clause 11, wherein: the second thigh orientation term is zero for user thigh orientation angles lower than zero, the second thigh orientation term is proportional to thigh orientation angle when the thigh orientation angle is within a first range of thigh orientation angles, the second thigh orientation term is defined by a decreasing linear relationship to approach a shank angle when the thigh orientation angle is within a second range of thigh orientation angles, the second range of thigh orientation angles being greater than the first range of thigh orientation angles, and the second thigh orientation term is equal to the shank angle when the thigh orientation angle is greater than the second range of thigh orientation angles.

Clause 13: the powered prosthesis of Clause 11 or Clause 12, wherein: the second thigh vertical acceleration term depends on a vertical acceleration of a user thigh with respect to gravity

Clause 14: the powered prosthesis of Clause 13, wherein the second thigh vertical acceleration term is determined by: determining a second double integral of a second quantity, the second quantity comprising a second factor subtracted from the vertical acceleration of the user thigh with respect to gravity; and multiplying the double integral by a second non-constant factor.

Clause 15: the powered prosthesis of Clause 14, wherein the second non-constant factor changes as a function of thigh orientation.

Clause 16: the powered prosthesis of Clause 15, wherein the second non-constant factor is constant for thigh orientations below a third thigh orientation threshold, and wherein, for thigh orientations that exceed the third thigh orientation threshold, the second non-constant factor is defined by a decreasing linear relationship that decreases linearly until reaching zero at a second predetermined offset from the third thigh orientation threshold.

Clause 17: the powered prosthesis of any one of Clauses 11 through 16, wherein the controller is configured to operate in a standing state or in a lifting state, and wherein the controller is configured to output the second signal configured to cause the ankle joint to move toward the target ankle angle when the lifting state is determined to be active.

Clause 18: the powered prosthesis of Clause 17, wherein the controller is configured to output the signal configured to cause the knee joint to move toward the target knee angle when the lifting state is determined to be active.

Clause 19: the powered prosthesis of Clause 17 or Clause 18, wherein the controller is configured to operate in the lifting state in response to detecting that a ground reaction force is below a threshold.

Clause 20: the powered prosthesis of Clause 19, wherein the controller is configured to operate in the standing state in response to detecting that the ground reaction force is above the threshold.

Clause 21: the powered prosthesis of Clause 20, wherein, when operating in the standing state, the controller is configured to output a third signal configured to cause application of a target knee torque at the knee joint, the target knee torque being determined based on a continuous function of knee position.

Clause 22: the powered prosthesis of Clause 20 or Clause 21, wherein, when operating in the standing state, the controller is configured to output a fourth signal configured to cause the ankle joint to move toward a target ankle equilibrium angle, the target ankle equilibrium angle being defined based on a linear relationship with knee position.

Clause 23: the powered prosthesis of any one of Clauses 20 through 22, wherein, at a transition from the lifting state to the standing state, the controller is configured to define a peak torque and an angle at which to apply the peak torque, the peak torque and the angle at which to apply the peak torque being defined based on a measured knee angle at the transition from the lifting state to the standing state.

Clause 24: the powered prosthesis of Clause 23, wherein, at the transition from the lifting state to the standing state, the controller is configured to set an ankle equilibrium angle as a measured ankle angle at the transition from the lifting state to the standing state.

Clause 25: a method for providing adaptive control of powered joint movement during climbing tasks, comprising: obtaining a thigh orientation term, a thigh angular velocity term, and a thigh vertical acceleration term based on sensor data, the sensor data being associated with a residual limb of a user; determining a target knee angle based on the thigh orientation term, the thigh angular velocity term, and the thigh vertical acceleration term; and outputting a signal configured to cause a knee joint to move toward the target knee angle.

Clause 26: one or more hardware storage devices storing instructions that are executable by one or more processors of a controller to configure the controller to: obtain a thigh orientation term, a thigh angular velocity term, and a thigh vertical acceleration term based on sensor data, the sensor data being associated with a residual limb of a user; determine a target knee angle based on the thigh orientation term, the thigh angular velocity term, and the thigh vertical acceleration term; and output a signal configured to cause a knee joint to move toward the target knee angle.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

What is claimed is:

1. A powered joint system configured to adaptively control powered joint movement during one or more movement tasks, the powered joint system comprising:
a knee joint;
one or more sensors configured to capture sensor data associated with a residual limb of a user; and
a controller comprising one or more processors and one or more hardware storage devices storing instructions that are executable by the one or more processors to configure the controller to:
obtain a thigh orientation term, a thigh angular velocity term, and a thigh vertical acceleration term based on the sensor data;
determine a target knee angle based on the thigh orientation term, the thigh angular velocity term, and the thigh vertical acceleration term; and
output a signal configured to cause the knee joint to move toward the target knee angle.

2. The powered joint system of claim 1, wherein the one or more movement tasks comprise one or more of stair climbing, squatting, lunging, or sit-to-stand transferring, and wherein the instructions are executable by the one or more processors to configure the controller to adaptively update the target knee angle based on updated sensor data, thereby enabling the controller to adapt to variable ascent height, user cadences, and/or user gait patterns.

3. The powered joint system of claim 1, wherein the thigh orientation term is proportional to an orientation of a user thigh with respect to gravity when a first thigh orientation threshold is satisfied.

4. The powered joint system of claim 3, wherein the thigh orientation term is set to zero when the first thigh orientation threshold is not satisfied.

5. The powered joint system of claim 1, wherein the thigh angular velocity term is proportional to a positive angular velocity of a user thigh.

6. The powered joint system of claim 1, wherein the thigh vertical acceleration term depends upon a vertical acceleration of a user thigh with respect to gravity.

7. The powered joint system of claim 6, wherein the thigh vertical acceleration term is determined by:
determining a double integral of a first quantity, the first quantity comprising a first factor subtracted from the vertical acceleration of the user thigh with respect to gravity; and
multiplying the double integral by a non-constant factor.

8. The powered joint system of claim 7, wherein the non-constant factor changes as a function of thigh orientation.

9. The powered joint system of claim 8, wherein the non-constant factor is constant for thigh orientations below a second thigh orientation threshold, and wherein, for thigh orientations that exceed the second thigh orientation threshold, the non-constant factor is defined by a decreasing linear relationship that decreases linearly until reaching zero at a predetermined offset from the second thigh orientation threshold.

10. The powered joint system of claim 1, further comprising an ankle joint.

11. The powered joint system of claim 10, wherein the instructions are executable by the one or more processors to configure the controller to:
obtain a second thigh orientation term and a second thigh vertical acceleration term based on the sensor data;
determine a target ankle angle based on the second thigh orientation term and the second thigh vertical acceleration term; and
output a second signal configured to cause the ankle joint to move toward the target ankle angle.

12. The powered joint system of claim 11, wherein:
the second thigh orientation term is zero for user thigh orientation angles lower than zero,
the second thigh orientation term is proportional to thigh orientation angle when the thigh orientation angle is within a first range of thigh orientation angles,
the second thigh orientation term is defined by a decreasing linear relationship to approach a shank angle when the thigh orientation angle is within a second range of thigh orientation angles, the second range of thigh orientation angles being greater than the first range of thigh orientation angles, and
the second thigh orientation term is equal to the shank angle when the thigh orientation angle is greater than the second range of thigh orientation angles.

13. The powered joint system of claim 11, wherein:
the second thigh vertical acceleration term depends on a vertical acceleration of a user thigh with respect to gravity.

14. The powered joint system of claim 13, wherein the second thigh vertical acceleration term is determined by:
determining a second double integral of a second quantity, the second quantity comprising a second factor subtracted from the vertical acceleration of the user thigh with respect to gravity; and
multiplying the double integral by a second non-constant factor.

15. The powered joint system of claim 14, wherein the second non-constant factor changes as a function of thigh orientation.

16. The powered joint system of claim 15, wherein the second non-constant factor is constant for thigh orientations below a third thigh orientation threshold, and wherein, for thigh orientations that exceed the third thigh orientation threshold, the second non-constant factor is defined by a decreasing linear relationship that decreases linearly until reaching zero at a second predetermined offset from the third thigh orientation threshold.

17. The powered joint system of claim 11, wherein the controller is configured to operate in a standing state or in a lifting state, and wherein the controller is configured to output the second signal configured to cause the ankle joint to move toward the target ankle angle when the lifting state is determined to be active.

18. The powered joint system of claim 17, wherein the controller is configured to output the signal configured to cause the knee joint to move toward the target knee angle when the lifting state is determined to be active.

19. The powered joint system of claim 17, wherein the controller is configured to operate in the lifting state in response to detecting that a ground reaction force is below a threshold.

20. The powered joint system of claim 19, wherein the controller is configured to operate in the standing state in response to detecting that the ground reaction force is above the threshold.

21. The powered joint system of claim 20, wherein, when operating in the standing state, the controller is configured to output a third signal configured to cause application of a target knee torque at the knee joint, the target knee torque being determined based on a continuous function of knee position.

22. The powered joint system of claim 20, wherein, when operating in the standing state, the controller is configured to output a fourth signal configured to cause the ankle joint to move toward a target ankle equilibrium angle, the target ankle equilibrium angle being defined based on a linear relationship with knee position.

23. The powered joint system of claim 20, wherein, at a transition from the lifting state to the standing state, the controller is configured to define a peak torque and an angle at which to apply the peak torque, the peak torque and the angle at which to apply the peak torque being defined based on a measured knee angle at the transition from the lifting state to the standing state.

24. The powered joint system of claim 23, wherein, at the transition from the lifting state to the standing state, the controller is configured to set an ankle equilibrium angle as a measured ankle angle at the transition from the lifting state to the standing state.

25. A method for providing adaptive control of powered joint movement during movement tasks, comprising:
obtaining a thigh orientation term, a thigh angular velocity term, and a thigh vertical acceleration term based on sensor data, the sensor data being associated with a residual limb of a user;
determining a target knee angle based on the thigh orientation term, the thigh angular velocity term, and the thigh vertical acceleration term; and
outputting a signal configured to cause a knee joint to move toward the target knee angle.

26. One or more hardware storage devices storing instructions that are executable by one or more processors of a controller to configure the controller to:
obtain a thigh orientation term, a thigh angular velocity term, and a thigh vertical acceleration term based on sensor data, the sensor data being associated with a residual limb of a user;
determine a target knee angle based on the thigh orientation term, the thigh angular velocity term, and the thigh vertical acceleration term; and
output a signal configured to cause a knee joint to move toward the target knee angle.
